(19) United States
(12) Patent Application Publication
(10) Pub. No.: US 2004/0063745 A1
(43) Pub. Date: Apr. 1, 2004

(54) 2-amino-4-(pyridin-2-yl)-thiazole derivatives as transforming growth factor beta (tgf-beta) inhibitors

(76) Inventors: Francoise Jeanne Gellibert, Les Ulis (FR); Charles David Hartley, Hertfordshire (GB); Neil Mathews, London (GB); James Michael Woolven, Hertfordshire (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/470,871

(22) PCT Filed: Jan. 30, 2002
(86) PCT No.: PCT/EP02/00940

(30) Foreign Application Priority Data
Feb. 2, 2001 (GB) 0102668.1

(51) Int. Cl. C07D 471/02; C07D 417/14; A61K 31/4745; A61K 31/4709

(52) U.S. Cl. 514/300; 514/314; 546/122; 546/167

(57) ABSTRACT

Therapeutically active thiazole derivatives of formula (I) wherein R¹, R², X and X′ are as defined in the specification, processes for the preparation thereof, the use thereof in therapy, particularly in the treatment or prophylaxis of disorders characterized by overexpression of transforming growth factor β(TAG-β), and pharmaceutical compositions for use in such therapy.

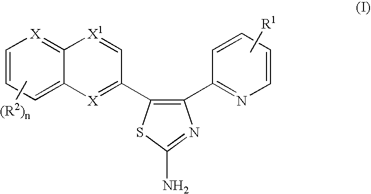

The present invention relates to novel thiazole derivatives, processes for the preparation thereof, the use thereof in therapy, particularly in the treatment or prophylaxis of disorders characterized by overexpression of transforming growth factor β(TAG-β), and pharmaceutical compositions for use in such therapy.

TAG-β is a multi-functional cytokine which belongs to the TAG-β super family which includes activisms/inhibits, bone morphogenetic proteins (BMPs) and TAG-βs. Three isoforms of TAG-β(TAG-β1, TAG-β2, and TAG-β3) have been identified in mammals, each of which is encoded by a distinct gene on different chromosomes (D. A. Lawrence, Eur. Cytokine. Netw., 1996, 7(3), 363). TAG-β initiates an intracellular signalling pathway which ultimately leads to the expression of genes that regulate cell cycle, control proliferative responses, or relate to extracellular matrix proteins that mediate cell adhesion, migration and intercellular communication. TAG-β has pleitropic effects including modulation of cell growth and differentiation, extracellular matrix formation, hematopoiesis, and immunomodulation (Roberts and Spoon, Handbook of Experimental Pharmacology, 1990, 95, 419-458).

A variety of cell surface proteins and receptors are known to transduce the signals initiated by the binding of the active TAG-β ligand to its receptors. Initiation of the TAG-β signalling pathway results from the binding of the TAG-β ligand to the extracelullar domain of the type II membrane receptor (Massague, Ann. Rev. Biochem., 1998, 67, 753.). The bound type II receptor then recruits type I (Alk5) receptor into a multimeric membrane complex, whereupon active type II receptor kinase phoshorylates and activates type I receptor kinase. The function of the type I receptor kinase is to phosphorylate a receptor-associated co-transcription factor, Smad-2 or Smad-3; thereby releasing it into the cytoplasm where it binds to Smad4. The PAI-1 gene is activated by TAG-β as a consequence of the abovementioned cellular pathway.

One approach to the treatment and/or prophylaxis of disorders characterized by the overexpression of TAG-β is inhibition of the TAG-β signal transduction. For example inhibition of the TAG-β type II receptor by overexpression of a dominant negative TAG-β type II receptor has previously been shown to prevent liver fibrosis and dysfunction in rat models (Proc. Natl. Acad. Sci, 1999, 96(5), 2345), and also to prevent progression of established liver fibrosis (Hepatology, 2000, 32, 247).

Pathological overexpression of TAG-β is known to be associated with a number of undesirable effects, leading ultimately to the development of serious pathogenic conditions (G. C. Blobe et al., N. Engl. J. Med., 2000, 1350). In particular, pathological overexpression of TAG-β may cause excessive accumulation of extracellular matrix (ECM), inhibition of cell proliferation and immunosupression. Excessive accumulation of ECM is known to lead to fibrotic diseases such as tumor fibrosis, radiation-induced fibrosis, fibrosis of the liver, kidney, lung, bowel, heart, pancreas, peritoneum or other organs. Fibrosis can lead to pathologic conditions such as cirrhosis, idiopathic pulmonary fibrosis, glomerulosclerosis and hypertrophic scars.

A number of other disease states are known to be associated with variations in the expression of genes which are controlled by TAG-β including cancer development, abnormal bone function and inflammatory disorders.

The development of compounds capable of inhibiting the TAG-β intracellular pathway is seen as a desirable way to effect prophylaxis and/or treatment of the above-mentioned conditions. Compounds capable of inhibiting the TAG-β intracellular pathway and/or the expression of TAG-β may be used in the treatment of disorders the symptoms of which often lead to the development of fibrotic conditions. For example, compounds of the present invention may be useful in treating the fibrosis associated with various liver-related conditions such as hepatitis B virus (HBV), hepatitis C virus (HCV), alcohol-induced hepatitis, haemochromatosis and primary biliary cirrhosis.

The compounds of the present invention are thiazole derivatives. Other thiazole compounds have previously been described for use in alternative medicinal applications. PCT Patent Application WO 96/03392 (Searle & Co) discloses a series of substituted thiazole compounds for the treatment of inflammation and inflammation-related disorders. WO 93/15071 (SmithKline Beecham Intercredit N.V.) describes a series of thiazolyl-pyridine derivatives which may be used as gastric acid secretion inhibitors. This type of compound may be useful in the treatment of gastrointestinal disorders such as gastric and duodenal ulcers, aspiration pneumonitis and Zollinger-Ellison Syndrome. U.S. Pat. No. 5,232,921 (Biziere et al.) discloses 2-alkylaminothiazoles having an affinity for muscarinic cholinergic receptors. None of the aforementioned patent applications describe the thiazole compounds of the present invention.

PCT Patent Application WO 00/12947 (Scios Inc.) describes the use of a series of quinazoline derivatives for treating various disorders associated with enhanced activity of kinase p38-α and/or TAG-β. The compounds described therein have been shown to inhibit the activities of both proteins and are therefore particularly useful for the treatment of conditions in which an enhanced activity towards both p38-α and TAG-β is required.

It has now been discovered that certain substituted thiazole compounds, as described below, are useful in the treatment or prophylaxis of disorders characterized by the overexpression of TAG-β. In particular, compounds of the present invention are TAG-β inhibitors which act at the TAG-β type I (Alk5) receptor level.

According to one aspect of the present invention, we provide compounds of formula (I),

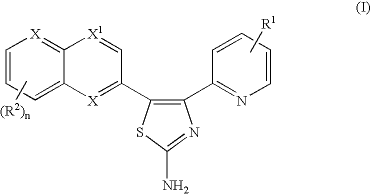

wherein,

R¹ is selected from H, halo (such as fluoro, chloro, bromo), —CN, —CF₃, Cl₁₋₄ alkyl or C₁₋₄ alkoxy;

n is selected from 0, 1, 2, 3, 4 or 5;

R², which may be the same or different, is selected from halo (such as fluoro, chloro, bromo), —CN, —CF₃, —OCF₃, C,₁₋₄ alkyl or C₁₋₄ alkoxy;

X is CH or N; and

X¹ is N when X is CH, and X¹ is CH when X is N;

and salts and solvates thereof (hereinafter “compounds of the invention”).

The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di- basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

Preferably, R¹ is positioned at the C(3) or C(6) position of the pyridine ring and is selected from H, halo (such as fluoro, chloro, bromo), —CN, —CF₃, C₁₋₄ alkyl or C₁₋₄ alkoxy. More preferably R¹ is H or C₁₋₄ alkyl. Alternatively, R¹ is more preferably H.

Preferably, n is 0 or 1.

It will be appreciated that the present invention is intended to include compounds having any combination of the preferred groups as listed hereinbefore.

Compounds of formula (I) which are of special interest as agents useful in the treatment or prophylaxis of disorders characterized by the overexpression of TAG-β are, 4-(Pyridin-2-yl)-5-quinolin-4-yl-1,3-thiazol-2-amine; 5-([1,5]Naphthyridin-2-yl)-4-pyridin-2-yl-1,3-thiazol-2-amine; and 4-(6-Methyl-pyridin-2-yl)-5-([1,5]naphthyridin-2-yl)-1,3-thiazol-2-amine, and salts and solvates thereof.

Compounds of formula (I) and salts and solvates thereof may be prepared by the methodology described hereinafter, constituting a further aspect of this invention. In yet a further aspect of the present invention there is provided a process for the preparation of intermediate compounds of formula (B) and (G). 4-Quinolinyl compounds of formula (I), in which X is CH and X¹ is N, may conveniently be prepared according to the general methodology in Scheme I below:

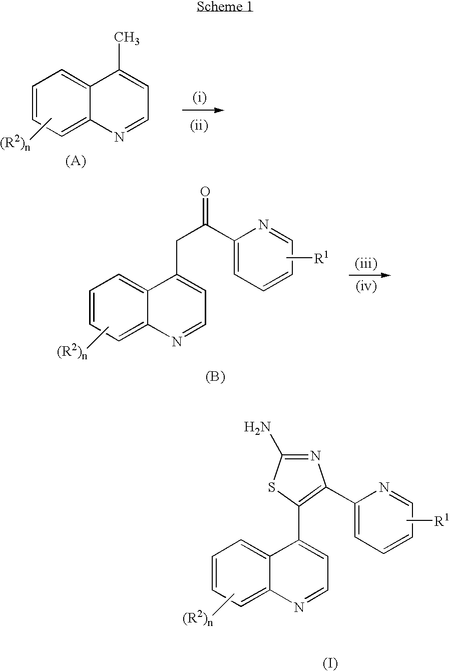

Reagents and conditions (preferred): (i) KHMDS, THF, −50° C.; (ii) R¹(C₅H₃N)CO₂Et, THF, −50° C.; (iii) polymer-supported pyridinium perbromide, THF, r.t.; (iv) thiourea, EtOH, reflux.

[1,5]Naphthyridine compounds of formula (i), in which X is N and X¹ is CH, may conveniently be prepared according to the general methodology in Scheme 2 below:

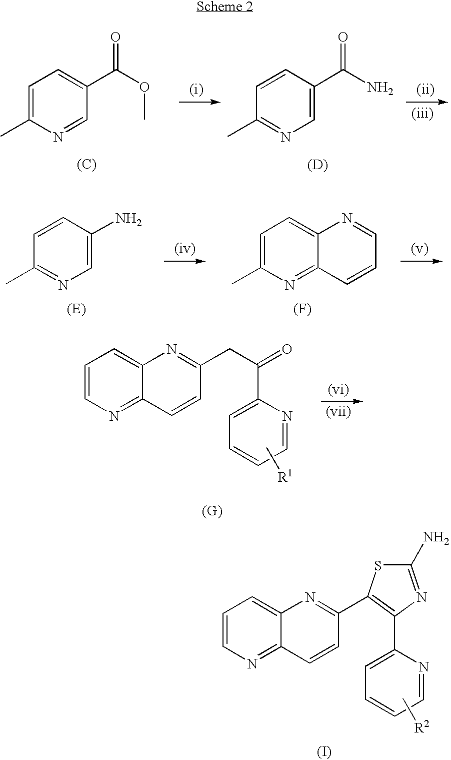

Reagents and conditions (preferred): (i) NH₄OH; (ii) Br₂, NaOH(aq); (iii) H₂SO₄ (conc), sodium m-nitrobenzenesulphonate, H₃BO₃, FeSO₄.7H₂O; (iv) glycerol, H₂O; (v) R³(C₅H₅N)CO₂Et, KHMDS, THF, −78° C.; (vi) Br₂, dioxan, r.t.; (vii) thiourea, 78° C.

List of Abbreviations

| | |
|---|---|
| EtOH | Ethanol |
| KHMDS | Potassium bis(trimethylsilyl)amide |
| THF | Tetrahydrofuran |

A general process according to the invention for preparing a 4-quinolinyl compound of formula (I) comprises:

(i) Addition of a suitable base, such as potassium bis(trimethylsilyl)amide or sodium bis(trimethylsilyl)amide to a substituted pyridine or quinoline of formula (A), preferably in the temperature range 0 to −75° C., more preferably in the temperature range −30 to −60° C., most preferably at −50° C., in the presence of a suitable solvent such as THF;

(ii) Addition of a suitable monosubstituted pyridyl ester, R¹(C₅H₃N)CO₂Et (wherein R¹ is hereinbefore defined) to the reaction mixture, preferably in the temperature range 0 to −75° C., more preferably in the temperature range −30 to −60° C., most preferably at −50° C., in the presence of a suitable solvent such as THF;

(iii) Halogenation of the resulting ketone (B) with a suitable halogenating agent, preferably a brominating reagent such as Br₂ or polymer-supported pyridinium perbromide, preferably in the temperature range 0-75° C., more preferably in the temperature range 20 to 60° C., most preferably at room temperature, in the presence of a suitable solvent such as THF; and

(iv) Addition of thiourea in a suitable solvent such as ethanol and heating the mixture under reflux.

Compounds of formula (A) may be prepared by processes analogous to those known in the art (e.g. R. H. F. Manske and M. Kulka, Org. React., 1953, 7, 59; Song et al, J. Heterocycl. Chem., 1993, 30,17). 6-Methyl-3-aminopyridine (E) may be prepared according to processes known in the art, for example, A. W. Hofmann, Ber. Dtsch. Ges., 1881, 14, 2725.

2-Methyl-[1,5]napthyridine (F) may be prepared according to processes known in the art, for example, Chem. Pharm. Bull., 1971, 19(9), 1857.

Monosubstituted pyridyl esters, R¹(C₅H₃N)CO₂Et (where R¹ is hereinbefore defined) as described in step (ii) above may be prepared by processes analogous to those known in the art. For example, where R¹=C(6)-OMe, Finger et al., J. Org. Chem., 1962, 27, 3965; where R¹=C(3)-OMe, Dejardin et al, Bull. Chem. Soc. Fr., 1979, 289; where R¹ =C(5)-Br, Chambers and Marfat, Synth. Commun., 1997, 27(3), 515; and where R¹=C(4)-CN, Heinisch and Lotsch, Heterocycles, 1987, 26(3), 731.

The compounds of the present invention have been found to inhibit phosphorylation of the Smad-2 or Smad-3 proteins by inhibition of the TAG-β type I (Alk5) receptor.

Accordingly, the compounds of the invention have been tested in the assays described herein and have been found to be of potential therapeutic benefit in the treatment and prophylaxis of disorders characterized by the overexpression of TAG-β.

Thus there is provided a compound of formula (I) or a physiologically acceptable salt or solvate thereof for use as a medicament in human or veterinary medicine, particularly in the treatment or prophylaxis of disorders characterized by the overexpression of TAG-β.

It will be appreciated that references herein to treatment extend to prophylaxis as well as the treatment of established conditions. It will further be appreciated that references herein to treatment or prophylaxis of disorders characterized by the overexpression of TAG-β, shall include the treatment or prophylaxis of TAG-β associated disease such as fibrosis, especially liver and kidney fibrosis, cancer development, abnormal bone function and inflammatory disorders, and scarring.

Other pathological conditions which may be treated in accordance with the invention have been discussed in the introduction hereinbefore. The compounds of the present invention are particularly suited to the treatment of fibrosis and related conditions.

Compounds of the present invention may be administered in combination with other therapeutic agents, for example antiviral agents for liver diseases, or in combination with ACE inhibitors or Angiotensin II receptor antagonists for kidney diseases.

According to another aspect of the invention, there is provided the use of a compound of formula (I) or a physiologically acceptable salt or solvate thereof for the manufacture of a medicament for the treatment and/or prophylaxis of disorders characterized by the overexpression of TAG-β, particularly fibrosis.

In a further aspect there is provided a method for the treatment of a human or animal subject with a disorder characterized by the overexpression of TAG-β, particularly fibrosis, which method comprises administering to said human or animal subject an effective amount of a compound of formula (I) or a physiologically acceptable salt or solvate thereof.

Compounds of the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

There is also provided according to the invention a process for preparation of such a pharmaceutical composition which comprises mixing the ingredients.

Compounds of the invention may, for example, be formulated for oral, buccal, parenteral, topical or rectal administration.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa butter or other glycerides.

Compounds of the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

Compounds of the invention may conveniently be administered in amounts of, for example, 0.01 to 100 mg/kg body weight, suitably 0.05 to 25 mg/kg body weight orally, one or more times a day. The precise dose will of course depend on the age and condition of the patient, the particular route of administration chosen, and is entirely within the discretion of the administering physician.

The following non-limiting Examples illustrate the present invention.

Intermediates

1-Pyridin-2-yl-2-quinolin-4-yl-ethanone

To a solution of Lepidine (9.54 g) in dry THF (100 ml) at −50° C. under argon, a solution of potassium bis-(trimethylsilyl)amide 0.5M in toluene (147 ml, 1.1 eq) was added dropwise. The solution was stirred at this temperature for 30 min, then a solution of ethyl picolinate (11.04 g) in dry THF (60 ml) was added and the reaction mixture was allowed to warm to r.t. overnight. The solvent was concentrated under reduced pressure and the solid precipitated with diethyl ether. The brown solid was then taken up in saturated NH₄Cl solution and the aqueous phase was extracted with ethyl acetate. The organic layer was dried over sodium sulfate and concentrated to give the title compound as an orange oil (12.83 g).

TLC SiO₂ CH₂Cl₂/MeOH 98/2 Rf 0.24

MS(API): 249 (MH+)

2-Methyl-[1,5]naphthyridine

A mixture of concentrated sulphuric acid (14 ml), sodium m-nitrobenzenesulphonate (11.30 g), boric acid (1.55 g, 0.039 mol) and iron sulphate heptahydrate (0.90 g, 3.23 mmol) was stirred at room temperature. Glycerol (8.0 ml) was added followed by 3-amino-6-methyl-pyridine (2.79 g, 0.025 mol) and water (14 ml). The resultant mixture was heated at 135° C. with stirring for 18 h. The reaction mixture was allowed to cool to room temperature, basified using 4N sodium hydroxide and the resultant mixture was extracted using ethyl acetate (×4). The extracts were combined and then preadsorbed onto silica gel (20 ml) prior to Biotage chromatography (using a 90 g silica gel cartridge) and eluting with ethyl acetate (neat). Appropriate fractions were combined and then evaporated to give the title compound (2.01 g, 55%) as a light brown cystalline solid. LC-MS (A4109272) Retention Time 2.06min M/Z 145=MH+

2-[1,5]Naphthyridin-2-yl-1-pyridin-2-yl-ethanone

To a stirred and cooled (−78° C.) solution of 2-methyl-[1,5]naphthyridine, (0.50 g, 3.46 mmol) and ethyl picolinate (0.52 g, 3.47 mmol) in anhydrous THF (30 ml) was added potassium hexamethyldimethylsilazide (0.5M solution in toluene) (13.9 ml, 6.94 mmol) dropwise over 10 minutes. This mixture was stirred at −78° C. for 1 h and then at room temperature for 20 h. Saturated aqueous ammonium chloride (100 ml) was added to the reaction mixture with stirring and the resultant mixture was partitioned between ethyl acetate and water. The aqueous phase was separated off and was extracted with ethyl acetate (×3). The extracts and organic phase were combined, washed with water and finally dried and evaporated to give the title compound (0.86 g) as an orange yellow solid.

[APCI MS] m/z 250 (MH+)

1-(6-Methyl-pyridin-2-yl)- 2-[1,5]naphthyridin-2-yl-ethanone

2-Methyl-[1,5]naphthyridine (4.34g, 30.1 mmol) and methyl-6-methyl picolinate (1.1eq, 5g, 33.11 mmol) were coupled as described for intermediate 2 to afford the title compound as an orange solid (6g).

[APCI MS] m/z 264 (MH+)

### EXAMPLES

### Example 1

4-(Pyridin-2-yl)-5-quinolin-4-yl-1,3-thiazol-2-amine

To a solution of 1-pyridin-2-yl-2-quinolin4-yl-ethanone (12.8 g) in THF (50 ml) was added polymer-supported pyridinium perbromide (Aldrich, 1 eq) and the suspension shaken overnight. The resin was removed by filtration, with the filtrate being added directly to thiourea (1 eq) and the resin washed many times with ethanol. The filtrate was heated at reflux for 4 h, allowed to cool and concentrated. The residue was taken up in EtOAc and washed with aqueous sodium carbonate. The organic phase was dried, concentrated and purified by chromatography on silica with CH₂Cl₂/MeOH 98:2+1% Et₃N as an eluant. The resulting solid was recrystallised (¹PrOH) to give the title compound as a pale yellow solid (8.2 g).

m.p 228° C.

TLC SiO₂ CH₂Cl₂/MeOH 90/10+Et₃N Rf 0.37

[APCI MS] m/z 305(MH+)

### Example 2

5-([1,5]Naphthyridin-2-yl)4-pyridin-2-yl-1,3-thiazol-2-amine

2-[1,5]Naphthyridin-2-yl-1-pyridin-2-yl-ethanone (0.20 g, 0.80 mmol) in dioxan (15 ml) was treated with bromine (0.050 ml, 0.97 mmol). The resultant orange suspension was stirred at room temperature for 1 hr and then thiourea (0.066 g, 0.88 mmol) was added and the resultant mixture was heated 78° C. with stirring for 4 h. The reaction was allowed to cool to room temperature and then an aqueous solution of ammonia (0.88M) (2 ml) was added with stirring. The resultant mixture was evaporated onto silica gel (˜10 ml) prior to Biotage chromatography (90 g silica gel cartridge) eluting with 5% methanol in ethyl acetate. Appropriate fractions were combined and then evaporated to give crude product as a brown gum. This gum was crystallised from ethyl acetate to give the title compound (0.11 g, 45%) as golden crystals.

[APCI MS] m/z 305 (MH+)

¹H NMR: (DMSO-d₆): δ8.87 (1H, dd, CH), 8.53 (1H, ddd, CH), 8.28 (1H, br.d, CH), 8.08 (1H, d, CH), 7.94 (1H, ddd, CH), 7.80 (1H, br.d, CH), 7.72 (1H, dd, CH), 7.56 (2H, br.s, —NH2), 7.50 (1 H, d, CH), 7.44 (1 H, ddd, CH).

### Example 3

4-(6-Methyl-pyridin-2-yl)-5-([1,5]naphthyridin-2-yl)-1,3-thiazol-2-amine

1-(6-Methyl-pyridin-2-yl)-2-([1,5]-naphthyridin-2-yl)-ethanone (131mg, 0.5mmol) was reacted with polymer-supported pyridinium perbromide (450mg, 0.5mmol) then with thiourea (76mg, 1mmol) as described for example 1, to afford the title compound as yellow crystals (27mg, 17%).

m.p: 188° C.

[APCI MS] 320 (MH+)

Biological Data

The compounds of Examples 1 to 3 were tested in vitro, using the biological assays described below. All of the compounds had an IC₅₀ value of 5 μM or below in Assay 1, and an IC₅₀ value of 1 μM or below in Assay 2.

Assays

Assay 1

The potential for compounds of the invention to inhibit TAG-β signalling may be demonstrated, for example, using the following in vitro assay.

The assay was performed in HepG2 cells stably transfected with the PAI-1 promoter (known to be a strong TAG-β responsive promoter) linked to a luciferase (firefly) reporter gene. The compounds were selected on their ability to inhibit luciferase activity in cells exposed to TAG-β. In addition cells were transfected with a second luciferase (Renilla) gene which was not driven by a TAG-β responsive promoter and was used as a toxicity control.

(96 well-)microplates are seeded, using a multidrop apparatus, with the stably transfected cell line at a concentration of 35000 cells per well in 200 μl of serum-containing medium. These plates are placed in a cell incubator.

18 to 24 hours later (Day 2), cell-incubation procedure is launched. Cells are incubated with TAG-β and a candidate compound (TAG-β inhibitor) at concentrations in the range 50 nM to 10 μM (final concentration of DMSO 1%). The final concentration of TAG-β(rhTGFβ-1) used in the test is 1 ng/mL. Cells are incubated with a candidate compound 15-30 mins prior to the addition of TAGβ. The final volume of the test reaction is 150 μl. Each well contains only one candidate compound and its effect on the PAI-1 promoter is monitored.

Columns 11 and 12 are employed as controls. Column 11 contains 8 wells in which the cells are incubated in the presence of TAGβ, without a candidate compound. Column 11 is used to determine the ‘reference TAG-β induced firefly luciferase value’ against which values measured in the test wells (to quantify inhibitory activity) may be compared. In wells A12 to D12, cells are grown in medium without TAG-β. The firefly luciferase values obtained from these positions are representive of the ‘basal firefly luciferase activity’. In wells E12 to H12, cells are incubated in the presence of TAG-β and 500 μM CPO (Cyclopentenone, Sigma), a cell toxic compound. The toxicity is revealed by decreased firefly and renilla luciferase activities (around 50% of those obtained in column 11).

12 to 18 hours later (day 3), the luciferase quantification procedure is launched. The following reactions are performed using reagents obtained from a Dual Luciferase Assay Kit (Promega). Cells are washed and lysed with the addition of 10 μl of passive lysis buffer (Promega). Following agitation (15 to 30 mins), luciferase activities of the plates are read in a dual-injector luminometer (BMG lumistar). For this purpose, 50 μl of luciferase assay reagent and 50 μl of ‘Stop & Glo’ buffer are injected sequentially to quantify the activities of both luciferases. Data obtained from the measurements are processed and analysed using suitable software. The mean Luciferase activity value obtained in wells A11 to H11 (Column 11, TAG-β only) is considered to represent 100% and values obtained in wells A12 to D12 (cells in medium alone) gives a basal level (0%). For each of the compounds tested, a concentration response curve is constructed from which an IC₅₀ value can be determined graphically.

Assay 2

The potential for compounds of the invention to inhibit the kinase Alk5 receptor may be demonstrated, for example, using the following in vitro assay.

The kinase domain of Alk5 was cloned and expressed in a baculovirus/Sf9 cells system. The protein (amino acids 162 to 503) was 6-His tagged in C-terminus. After purification by affinity chromatography using a Ni²⁺column, the autophosphorylation was tested. The enzyme was incubated in a medium containing:Tris 50 mM pH 7.4; NaCl 100 mM; MgCl₂ 5 mM; MnCl₂ 5 mM; DTT 10 mM. The enzyme was preincubated with the compounds (0.1% DMSO final in the test) 10 minutes at 37° C. The reaction was initialised by the addition of 3 μM ATP (0.5 μCi gamma-33P-ATP). After 15 minutes at 37° C. the reaction was stopped by addition of SDS-PAGE sample buffer (50 mM Tris-HCl, pH 6.9, 2.5% glycerol, 1% SDS, 5% beta-mercaptoethanol). The samples were boiled for 5 minutes at 95° C. and run on a 12% SDS-PAGE. The dried gels were exposed to a phosphor screen over-night. Alk5 autophosphorylation was quantified using a STORM (Molecular Dynamics).

1. A compound of formula (I), 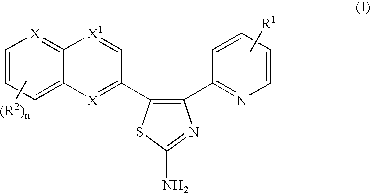
wherein,
R¹ is selected from H, halo, —CN, —CF₃, C₁₋₄ alkyl or C₁₋₄ alkoxy;
n is selected from 0, 1, 2, 3, 4 or 5;
R², which may be the same or different, is selected from halo, —CN, —CF₃, —OCF₃, C₁₋₄
alkyl or C₁₋₄ alkoxy;
X is CH or N; and
X¹ is N when X is CH, and X¹ is CH when X is N;
and salts and solvates thereof.

2. A compound of formula (I) as claimed in claim 1 wherein R¹ is positioned at the C(3) or C(6) position of the pyridine ring and is selected from H, halo, —CN, —CF³, C₁₋₄ alkyl or C₁₋₄ alkoxy.

3. A compound of formula (I) as claimed in claim 2, wherein R¹ is H or C₁₋₄ alkyl.

4. A compound of formula (I) as claimed in any one of claims 1 to 3 wherein n is 0 or 1.

5. A compound of formula (I) as claimed in claim 1 selected from:
4-(Pyridin-2-yl)-5-quinolin-4-yl-1,3-thiazol-2-amine;
5-([1,5]Naphthyridin-2-yl)-4-pyridin-2-yl-1,3-thiazol-2-amine; and
4-(6-Methyl-pyridin-2-yl)-5-([1,5]naphthyridin-2-yl)-1,3-thiazol-2-amine,
and salts and solvates thereof.

6. A pharmaceutical composition comprising a compound of formula (I) as claimed in any one of claims 1 to 5, together with a pharmaceutically acceptable diluent or carrier.

7. A compound of formula (I) as claimed in any one of claims 1 to 5, for use as a medicament.

8. The use of a compound as claimed in any one of claims 1 to 5 in the manufacture of a medicament for the treatment and/or prophylaxis of a disorder characterized by the overexpression of TAG-β.

9. A method for the treatment of a human or animal subject with a disorder characterized by the overexpression of TAG-β, which method comprises administering to said human or animal subject an effective amount of a compound of formula (I) as claimed in any one of claims 1 to 5 or a physiologically acceptable salt or solvate thereof.

10. A process for the preparation of a compound of formula (I), 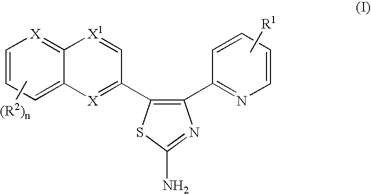
wherein,
R¹ is selected from H, halo, —CN, —CF₃, C₁₋₄ alkyl or C₁₋₄ alkoxy; n is is an integer selected from 0, 1, 2, 3, 4 or 5; R², which may be the same or different, is selected from halo, —CN, —CF₃, —OCF₃, C₁₋₄ alkyl or C₁₋₄ alkoxy; X is CH; and X¹ is N; and salts and solvates thereof,
which process comprises:
a) addition of a suitable halogenating agent to a compound of formula (B), 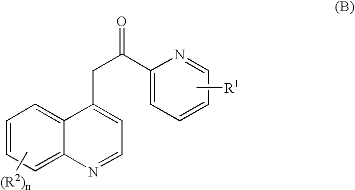
where R¹ and R² are defined above; and
(b) subsequent addition of thiourea to the resulting reaction mixture.

11. A process for the preparation of a compound of formula (I), 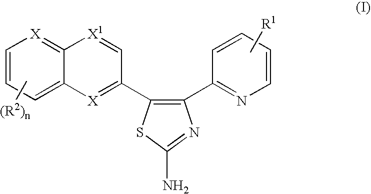
wherein, R¹ is selected from H, halo, —CN, —CF₃, C₁₋₄ alkyl or C₁₋₄ alkoxy; n is is an integer selected from 0, 1, 2, 3, 4 or 5; R², which may be the same or different, is selected from halo, —CN, —CF₃, —OCF₃, C₁₋₄ alkyl or C₁₋₄ alkoxy; X is N; and X¹ is CH; and salts and solvates thereof,
which process comprises:
a) addition of bromine to a compound of formula (G): 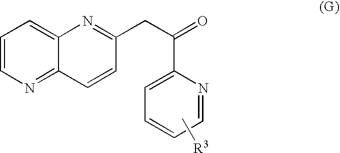
where R³ is defined above; and
b) subsequent addition of thiourea to the resulting reaction mixture.
